(19) United States
(12) Patent Application Publication
(10) Pub. No.: US 2026/0337229 A1
(43) Pub. Date: Oct. 29, 2026

(54) OXYTOCIN DERIVATIVES AND METHODS OF USE

(71) Applicant: Seattle Children's Hospital (d/b/a Seattle Children's Research Institute), Seattle, WA (US)

(72) Inventors: Alex Kiselyov, Los Angeles, CA (US); Christian L. Roth, Seattle, WA (US); Clinton T. Elfers, Seattle, WA (US)

(21) Appl. No.: 19/104,431

(22) PCT Filed: Aug. 23, 2023
(86) PCT No.: PCT/US2023/072729

§ 371 Date: Feb. 18, 2025

Related U.S. Application Data
(60) Provisional application No. 63/373,280, filed on Aug. 23, 2022.

(51) Int. Cl. C07K 7/16; A61K 9/08; A61K 38/00; A61K 47/40; A61P 3/04

(52) U.S. Cl. CPC C07K 7/16; A61K 9/08; A61K 47/40; A61P 3/04; A61K 38/00

(57) ABSTRACT

Provided herein are compounds of formula (I). In certain embodiments, the compounds of the disclosure are analogs of oxytocin. In certain embodiments, the compounds of the disclosure have significantly larger half-lives than oxytocin, and are useful in the treatment of obesity and in the reduction of appetite or food intake in a subject.

### CROSS-REFERENCE TO RELATED APPLICATION

This application claims priority to U.S. Provisional Patent Application No. 63/373,280 entitled “OXYTOCIN DERIVATIVES AND METHODS OF USE,” filed Aug. 23, 2022, the disclosure of which is incorporated herein by reference in its entirety.

### STATEMENT REGARDING FEDERALLY SPONSORED RESEARCH

This invention was made with government support under DK120236 awarded by the National Institutes of Health. The government has certain rights in the invention.

### BACKGROUND

Obesity and its comorbidities cause significant, yet preventable, morbidity and mortality and are major public health burdens worldwide. Diet and exercise alone are not sufficient to produce long term decreases in weight, as many studies have demonstrated weight regain after one year of initial weight loss. Although drug interventions can improve weight loss, treatments often have limited long-term efficacy and are associated with safety and tolerability issues with little or no impact on disease reversal.

Oxytocin (OXT) has emerged as an attractive target for treating obesity. There is a growing body of evidence that peripheral administration of OXT reduces food intake, enhances energy expenditure, and prevents or reduces weight gain in obese rodent models and non-human primates. This is thought to occur despite the reduced ability of OXT to penetrate into the brain from the periphery, with small amounts of OXT being detected in the CSF. Instead, the mechanism of OXT-related reductions in food intake is thought to occur, in part, through activation of the vagal afferent neurons.

In humans, the use of intranasal administration as a non-invasive approach to bypass the blood-brain barrier has become increasingly popular in disease treatment. Indeed, intranasal OXT administration has been shown to reduce body weight in obese humans. However, some of these treatments require multiple administrations daily, making adherence to drug regimens often difficult. As a consequence, long-term effects of OXT treatments are not well established in humans.

Thus, there is an unmet need for pharmaceutical agents that have longer half-lives than OXT, as well as potency, and selectivity. The present invention solves this unmet need.

### BRIEF SUMMARY OF THE INVENTION

In various aspects, a compound of formula (I), or a pharmaceutically acceptable salt, enantiomer, diastereomer, or solvate thereof, is provided. The compound of formula (I) has the structure:

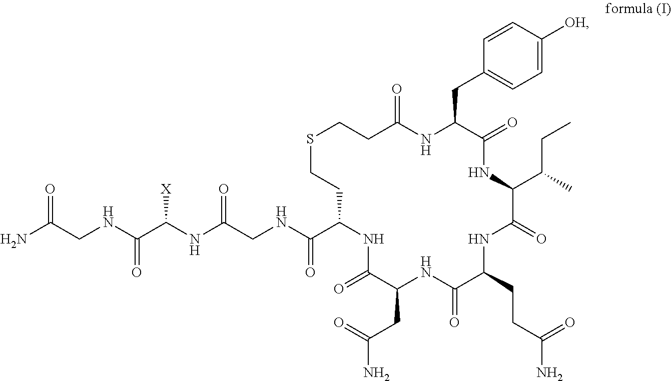

wherein:
X has the structure

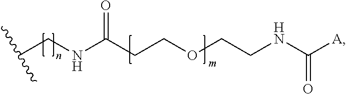

A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

In various aspects, a composition having the compound of formula (I), or a pharmaceutically acceptable salt, enantiomer, diastereomer, or solvate thereof, and at least one cyclodextrin is provided.

In various aspects, a method of treating, ameliorating, or preventing obesity in a subject is provided. The method includes administering to the subject a therapeutically effective amount of a compound of formula (I), or a pharmaceutically acceptable salt, enantiomer, diastereomer, or solvate thereof.

### BRIEF DESCRIPTION OF THE FIGURES

The drawings illustrate generally, by way of example, but not by way of limitation, certain embodiments of the present application.

The patent or application file contains at least one drawing executed in color. Copies of this patent or patent application publication with color drawing(s) will be provided by the Office upon request and payment of the necessary fee.

FIG. 1 illustrates the structure of long-acting oxytocin analogue ASK2131. The native oxytocin peptide was modified with a substitution of the Leu8 to a Lys appended with a polyethylene glycol space and a palmitoyl group and with a substitution of Gly for the Pro7 to enhance selectivity for the OXT receptor to create the prior peptide ASK1476. The design of ASK1476 had been further enhanced by replacing its disulfide bridge with a thioether isostere and removing the Cys amino group, to create ASK2131.

FIGS. 2A-2E show changes in body weight, food intake, and water intake were tested during 22-day treatment with ASK2131 vs. pair-feeding to ASK2131 vs. OXT vs. vehicle in DIO male Sprague Dawley rats which were matched based on baseline food intake and body weight gain trajectory. Body weight was reduced in all treatment groups with strongest reductions in percent reductions of body weight (FIGS. 2A, 2B), and total body weight (FIG. 2C) in rats treated with ASK2131, followed by pair-fed to ASK2131. Daily food intake was reduced during the whole duration of 22-day treatment with ASK2131 in comparison to vehicle (FIG. 2D). Water intake was comparable between all treatment groups (FIG. 2E).

FIGS. 2A, 2D: Data were analyzed by repeated measure two-way ANOVA and Šidák's correction for multiple comparisons for comparing treatment groups vs. vehicle. #/##p<0.05/0.01 ASK2131 vs. OXT. */**p<0.05/0.01 ASK2131 vs. vehicle. FIGS. 2B, 2C: Analysis by one way ANOVA followed by Šidák's multiple comparisons test. *p<0.05, **p<0.01, ***p<0.001, ****p<0.0001.

FIGS. 3A-3B show an analysis of food intake pattern of continuously automatically recorded food intake of singly housed rats in BioDAQ cages. Strong reductions of food intake were noted already at days 1 and 2 during treatment with ASK2131, but not to OXT compared to baseline, both drugs at 50 nmol/kg (FIG. 3A). Cumulative food intake throughout the whole experiment was more reduced in response to ASK2131 than to oxytocin, even when doses were increased for oxytocin every 2 days up to 600 nmol/kg while the dose for ASK2131 was kept at 50 nmol/kg/d (FIG. 3B). A—Vehicle; C—Oxytocin; B—ASK2131. Light 9-10%—light cycle, light 0-2%—dark cycle.

FIGS. 4A-4C show changes of weight (FIG. 4A), food intake (FIG. 4B), and water intake (FIG. 4C) in DIO rats treated with ASK2131 after 6 days of baseline measures, starting dose 1 nmol/kg/day, then increase to 2, 5, 10, 20, 50, and finally 100 nmol/kg every 2 days. The low dose of ASK2131 was effective in significantly reducing body weight and food intake, higher doses did not result in greater reductions of food intake and body weight. Analysis by Two-Way ANOVA test with Šidák's multiple comparisons test. *p<0.05 vs. baseline, **p<0.01 vs. baseline, ***p<0.001 vs. baseline.

FIGS. 5A-5D show quantitative real-time PCR results for gene expression of thermoregulatory proteins. Inguinal white adipose tissue (IWAT) and interscapular brown adipose tissue (IBAT) were collected from diet-induced obese rats treated with vehicle, oxytocin, ASK2131, and the pair-fed to the ASK2131-treated rats. Gene expression (mRNA levels) determined by qRT-PCR for thermogenetic markers UCP1 (IWAT: FIG. 5A; IBAT: FIG. 5B) and Cox8b (IWAT: FIG. 5C; IBAT: FIG. 5D). Individual data points are indicated (o); *p<0.05. Significantly increased gene expression of UCP-1 or Cox8b versus vehicle treated controls means stimulation of thermogenesis suggesting stimulated energy expenditure.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve, or affect a condition contemplated herein, or the symptoms of a condition contemplated herein. Such treatments may be specifically tailored or modified, based on knowledge obtained from the field of pharmacogenomics.

### Oxytocin Derivatives

Compounds of formula (I) or otherwise described herein can be prepared by the general schemes described herein, using the synthetic method known by those skilled in the art. The following examples illustrate non-limiting embodiments of the compound(s) described herein and their preparation.

In certain embodiments, a compound of formula (I), or a pharmaceutically acceptable salt, enantiomer, diastereomer, or solvate thereof is provided:

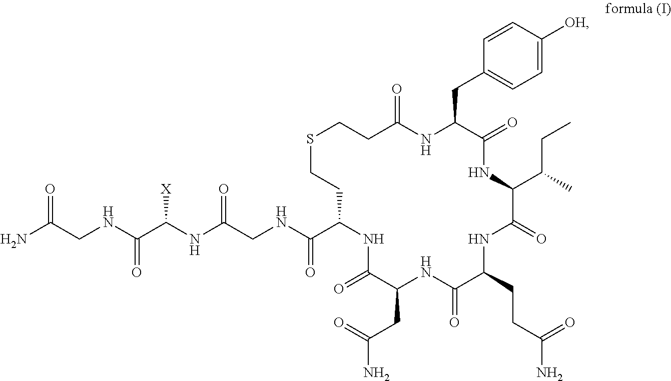

wherein:

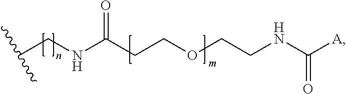

X has the structure
A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

In certain embodiments, n is 1. In certain embodiments, n is 2. In certain embodiments, n is 3. In certain embodiments, n is 4. In certain embodiments, n is 5. In certain embodiments, n is 6. In certain embodiments, n is 2, 3, or 4.

In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15. In certain embodiments, m is 16. In certain embodiments, m is 17. In certain embodiments, m is 18. In certain embodiments, m is 19. In certain embodiments, m is 20. In certain embodiments, m is 4, 5, 6, 7, 8, 9, or 10.

In certain embodiments, A is C₁₀₋₂₀ alkyl. In certain embodiments, A is C₁₀ alkyl. In certain embodiments, A is C₁₁ alkyl. In certain embodiments, A is C₁₂ alkyl. In certain embodiments, A is C₁₃ alkyl. In certain embodiments, A is C₁₄ alkyl. In certain embodiments, A is C₁₅ alkyl. In certain embodiments, A is C₁₆ alkyl. In certain embodiments, A is C₁₇ alkyl. In certain embodiments, A is Cis alkyl. In certain embodiments, A is C₁₉ alkyl. In certain embodiments, A is C₂₀ alkyl. In certain embodiments, A is C₂₁ alkyl. In certain embodiments, A is C₂₂ alkyl. In certain embodiments, A is C₂₃ alkyl. In certain embodiments, A is C₂₄ alkyl. In certain embodiments, A is C₂₅ alkyl. In certain embodiments, when A is a C₁₀₋₂₀ alkyl it is a straight C₁₀₋₂₀ alkyl. In certain embodiments, when A is a C₁₀₋₂₀ alkyl it is a branched alkyl.

In certain embodiments, the compound is

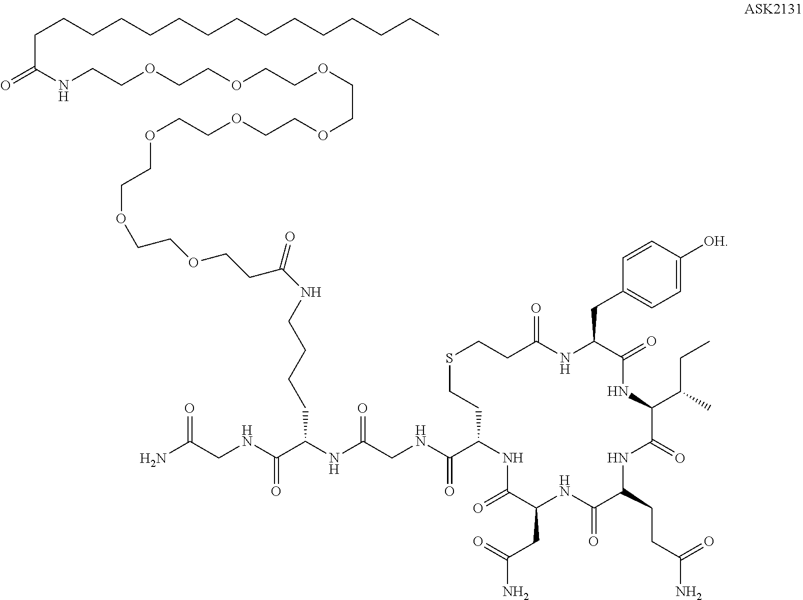

Also contemplated are all combinations and/or permutations of n, m, and A with each other, as defined herein.

In certain embodiments, a composition including the compound of formula (I) is provided. In certain embodiments, the composition includes at least one cyclodextrin and a compound of formula (I) as described herein. The composition can be a pharmaceutical composition or formulation suitable for administration to subjects. In certain embodiments, the composition is a solid composition containing lyophilized compound of formula (I). Solid compositions, in some embodiments, can be reconstituted with sterile solution rendering them suitable for injection by any suitable means described herein. Reconstitution can include dissolving the compound of formula (T) into solution. In certain embodiments, the composition is a liquid composition. The liquid composition can be a solution in which the compound of formula (I) is dissolved. Liquid compositions can be formulated as injectable formulations or as aerosol formulations suitable for intranasal administration. Any of the compositions described herein can also be formulated in unit dose form.

In certain embodiments, any of the compositions or formulations described herein can contain at least one additional pharmaceutically acceptable excipient or carrier as described herein. In certain embodiments, the at least one additional pharmaceutically acceptable excipient or carrier does not individually, collectively, or in combination with the compound of formula (I) exhibit any appetite-suppressing activity or cause reduction in weight in a subject when administered, i.e. the compound of formula (I) is the only pharmaceutically active ingredient.

As noted herein, the compositions can include one or more cyclodextrins. Suitable cyclodextrins (CD) include, but are not limited to, α-cyclodextrin, O-cyclodextrin, γ-cyclodextrin, hydroxyethyl-β-CD, hydroxypropyl-β-CD, sulfobutylether-β-CD, methyl-β-CD, dimethyl-β-CD, randomly 12 emethylated-β-CD, randomly methylated-β-CD, carboxymethyl-β-CD, carboxymethyl ethyl-β-CD, diethyl-β-CD, tri-O-methyl-β-CD, tri-O-ethyl-β-CD, tri-O-butyryl-β-CD, tri-O-valeryl-β-CD, tri-O-hexanoyl-β-CD, glucosyl-β-CD, maltosyl-β-CD, 2-hydroxy-3-trimethyl-ammoniopropyl-β-CD, and/or combinations thereof. In certain embodiments, the at least one cyclodextrin is hydroxypropyl-β-cyclodextrin.

The at least one cyclodextrin and the compound of formula (I) can be in a ratio of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or about 20:1 (wt/wt). In certain embodiments, when more than one cyclodextrin is used, each can be independently present in any of the ratios described herein with respect to cyclodextrin and the compound of formula (I). In certain embodiments, each compound of formula (I) is present in amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% (w/w) relative to the amount of cyclodextrin.

In certain embodiments, the compound of formula (I) is the only active agent in the pharmaceutical compositions described herein.

In a non-limiting exemplification, OXT analog ASK2131 was tested for its effect on food intake and weight gain in lean and DIO Sprague-Dawley rats. In vitro function assays were conducted to assess its ability to bind to the oxytocin and vasopressin (V₁ₐR and V₂R) receptors. In addition, pharmacokinetics of this peptide was examined following subcutaneous injection.

For the long-term study, the illustrative dose of 50 nmol/kg was chosen based on pilot experiments before starting this experiment. While this dose demonstrates about 25-30% decline in food intake, the first week of treatment showed closer to 40% decrease. This overtime increased to around 25-30%. Comparison of ASK2131-treated rats to their pair fed counterparts demonstrated that the weight loss due to ASK2131 was not driven solely by the reduction in food intake. Studies in non-human primates have shown that OXT elicits weight loss through increases in energy expenditure. Conversely, reduced OXT signaling is associated with obesity and reduced energy expenditure and thermogenesis in BAT. Without wishing to be limited by any theory, systemic OXT administration can in certain embodiments elevate the gene expression of uncoupling protein-1 (UCP-1) in inguinal white adipose tissue (IWAT) of DIO rats.

In this study, the lack of stimulation of uncoupling protein-1 (UCP1) and Cox8b mRNA levels in IWAT and IBAT might indicate that ASK2131 does not have a major impact on stimulation of thermogenesis by these molecular mechanisms in contrast to OXT. Studies in non-human primates have shown that OXT elicits weight loss through increases in energy expenditure. Conversely, reduced OXT signaling is associated with obesity and reduced energy expenditure and thermogenesis in brown adipose tissue (BAT). Without being bound by theory, it is believed that systemic OXT might elevate the gene expression of UCP-1 in IWAT of DIO rats.

To test the full range of dose responsiveness, a dose escalation study was completed on DIO Sprague-Dawley rats, as obese rodents appear to be more sensitive to OXT than their lean counter parts. In addition, the effect of long-term exposure to ASK2131 was examined by treating DIO Sprague-Dawley rats for 22 days with this peptide in comparison to native OXT. At the low dose of 2 nmol/kg (0.0011 mg), a 47% decrease in food intake was observed in the DIO Sprague-Dawley rats compared to their baseline food intake. To the best of the inventors' knowledge, this is the most profound food intake reduction observed by an OXT analog to date. The dose response curve to ASK2131 was U-shaped. This U-shaped curve was evident in both the lean and DIO dose escalation studies. At 2 nmol/kg, rats had already shown an ~4% decrease on body weight compared to baseline after only two treatments. This is of particular note when compared to studies examining the effect of OXT on food intake and body weight. In a study using non-human primates, a 3.3% change in body weight was observed after one month of treatment with OXT, two-weeks at 0.2 mg/kg followed by two-weeks at 0.4 mg/kg. In DIO rats, a single injection of 1000 μg (1000 nmol) of OXT led to a 12% decrease in food intake and a modest 0.01% change in body weight after a single dose.

These doses, up to 500× the dose of ASK2131 used, had modest, yet significant effects on food intake and body weight. This U-shaped response has also been observed in human trials using intranasal OXT. The present results confirm the U-shaped response found in a prior study testing the OXT analogue ASK1476. Without being bound by theory, this phenomenon is likely a result of OXT receptor internalization. Prolonged OXT treatment has been shown to promote desensitization of the OXT receptors in vitro and reductions of OXTR binding.

Alternatively, and without being bound by theory, it is possible that at higher doses, OXT may have lower weight loss efficacy due to opposing actions at the V1bR. Activation of the V1bR increases ACTH release, which may in turn increase drive to eat and body, weight and single nucleotide polymorphisms of V1bR have been linked to higher body mass index. In addition, U-shaped responses were seen in response to single OXT doses. In a neurobehavioral study studying acute fear responses, the OXT-induced inhibition of amygdala responses to fear revealed a significant decrease in amygdala activation assessed by functional neuroimaging following 24 IU doses, but not lower (12 IU) or higher (48 IU) doses.

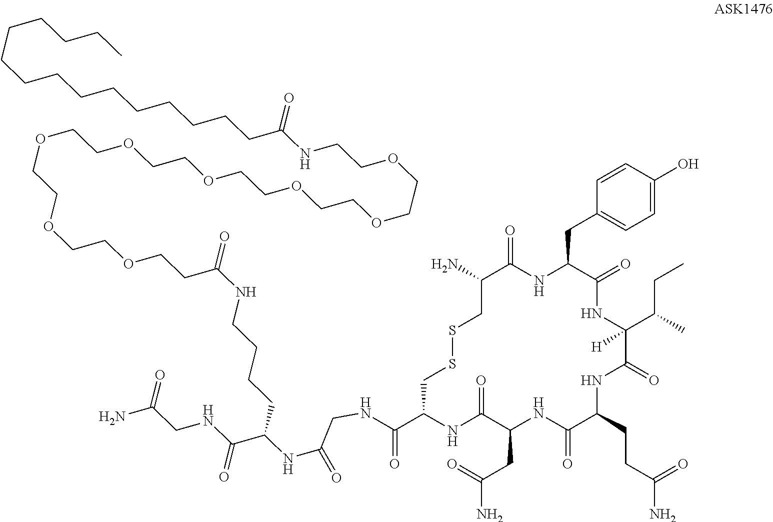 [SMILES: CCCCCCCCCCCCCCCC(=O)NCCOCCOCCOCCOCCOCCOCCOCCOCCC(=O)NCCCC[C@H](NC(=O)CNC(=O)[C@@H]1CSSC[C@H](N)C(=O)N[C@@H](Cc2ccc(O)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

Without being bound by theory, a variety of structural features in the compounds of formula (I), such as ASK2131, are believed to result in improved pharmaceutical properties. Features of ASK2131 compared to those in OXT include replacement of the disulfide bridge with a thioether isostere and elimination of the Cys amino group. In addition, ⁷Pro was replaced with ⁷Gly to improve V1aR selectivity and ⁸Leu was changed to ⁸Lys followed by modification of terminal NH₂ with polyethylene glycol spacer and a lipid palmitoyl group, in some embodiments. In certain embodiments and without being bound by theory, the hydrophobic A group described herein, such as the palmitoyl group enhances the plasma proteins binding of the resulting molecule and decreases the free fraction of the molecule available for metabolism leading to the overall increase of ASK2131 half-life in vivo, and other compounds of formula (I).

Examination of receptor pharmacology and pharmacokinetics for ASK2131 compared to ASK1476 and OXT have no indication that ASK2131 should display enhanced performance, and consequently the present findings and use of the “X” group as defined herein are surprising and unexpected. ASK2131 is still a potent activator of V₂R, with an EC₅₀ of 0.36 nM in the cAMP assay. No adverse effects caused by the activation of V₂R were observed. The replacement of the disulfide bridge with a thioether isostere improves the stability of disulfide-rich polypeptides. This improved stability is at least in part responsible for the improved function of ASK2131. OXT does not cross the blood-brain barrier (BBB), instead it is effluxed from the brain into the periphery using PTS-1. Recent evidence suggests a RAGE mediated mechanism of OXT transport to the brain. Obesity is known to increase expression of RAGE, suggesting that the DIO Sprague-Dawley rats described herein had increased transport.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

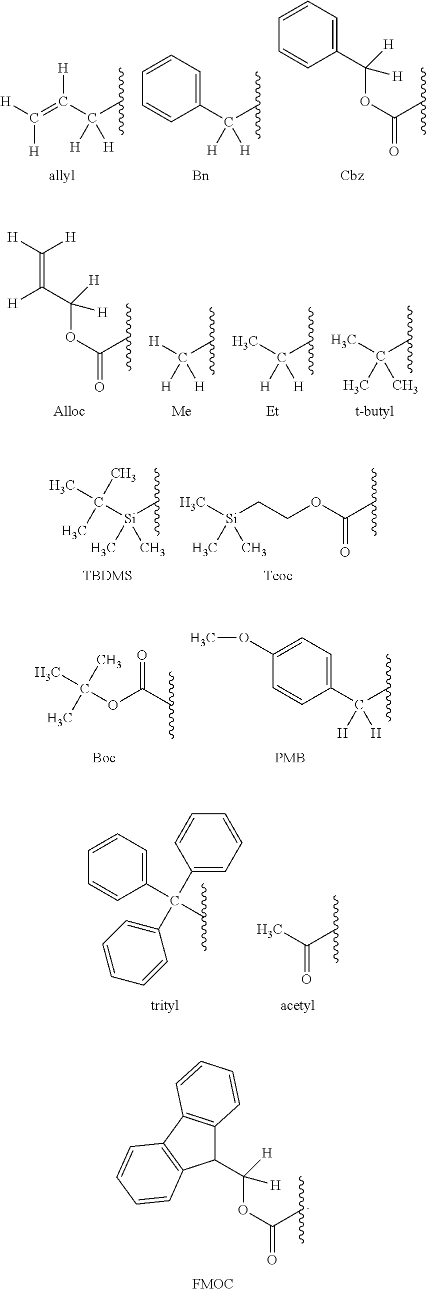

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Pharmacology

In certain embodiments, the compound(s) described herein can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In certain embodiments, the compound(s) described herein can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg. 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment or Amelioration

The disclosure includes a method of treating, preventing, and/or ameliorating obesity using the compounds of the disclosure. The disclosure includes a method of reducing appetite or food intake using the compounds of the disclosure. Compounds of the disclosure have demonstrated profound reductions in a subject's food intake and body weight.

Compounds of the disclosure can also be used to treat, reduce, and/or ameliorate symptoms of postpartum hemorrhage and incomplete or inevitable abortion. Compounds of the disclosure can also be used to induce labor (childbirth), for the initiation or improvement of uterine contractions. In certain embodiments, compounds of the disclosure can be used in subjects with a medical indication for initiation of labor, such as individuals that have Rh problems, maternal diabetes, preeclampsia at or near term, and the like. Compounds of the disclosure can be used to induce or produce uterine contractions during the third stage of labor. Compounds of the disclosure can also be used to treat or ameliorate sexual dysfunction in men or women. In certain embodiments, compounds of the disclosure can be used to treat, ameliorate, or prevent any physiological condition that can be treated, ameliorated, or prevented by oxytocin.

The methods described herein include administering to the subject a therapeutically effective amount of at least one compound described herein, which is optionally formulated in a pharmaceutical composition. In certain embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats obesity or decreases appetite or food intake.

In certain embodiments, administering the compound(s) described herein to the subject allows for administering a lower dose of the additional therapeutic agent as compared to the dose of the additional therapeutic agent alone that is required to achieve similar results in treating obesity or decreasing appetite or food intake in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

In certain embodiments, the therapeutically effective amount for treating, preventing, and/or ameliorating obesity using the compounds of the disclosure is from 0.1 to 100 nmol/kg/day. In certain embodiments, the therapeutically effective amount for treating, preventing, and/or ameliorating obesity using the compounds of the disclosure is about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nmol/kg/day, or any range between any of these values. In some embodiments, the therapeutically effective amount is dosed multiple times per day, such as two, three, four, or more times per day.

In certain embodiments, the therapeutically effective amount for treating, reducing, and/or ameliorating symptoms of postpartum hemorrhage and incomplete or inevitable abortion using the compounds of the disclosure is from 0.001 to 100 nmol/kg/day. In certain embodiments, the therapeutically effective amount for treating, reducing, and/or ameliorating symptoms of postpartum hemorrhage and incomplete or inevitable abortion using the compounds of the disclosure is about 0.001, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nmol/kg/day, or any range between any of these values. In some embodiments, the therapeutically effective amount is dosed multiple times per day, such as two, three, four, or more times per day.

In certain embodiments, the therapeutically effective amount for treating, reducing, and/or ameliorating symptoms of postpartum hemorrhage and incomplete or inevitable abortion using the compounds of the disclosure is from 0.001 to 1000 nmol/kg/day. In certain embodiments, the therapeutically effective amount for treating, reducing, and/or ameliorating symptoms of postpartum hemorrhage and incomplete or inevitable abortion using the compounds of the disclosure is about 0.001, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1000 nmol/kg/day, or any range between any of these values. In some embodiments, the therapeutically effective amount is dosed multiple times per day, such as two, three, four, or more times per day.

In certain embodiments, the therapeutically effective amount for inducing labor (childbirth) and/or for the initiation or improvement of uterine contractions using the compounds of the disclosure is from 0.001 to 1000 nmol/kg/day. In certain embodiments, the therapeutically effective amount for inducing labor (childbirth) and/or for the initiation or improvement of uterine contractions using the compounds of the disclosure is about 0.001, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1000 nmol/kg/day, or any range between any of these values. In some embodiments, the therapeutically effective amount is dosed multiple times per day, such as two, three, four, or more times per day.

In certain embodiments, the therapeutically effective amount for treating, preventing, and/or ameliorating sexual dysfunction in men or women using the compounds of the disclosure is from 0.001 to 1000 nmol/kg/day. In certain embodiments, the therapeutically effective amount for treating, preventing, and/or ameliorating sexual dysfunction in men or women using the compounds of the disclosure is about 0.001, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1000 nmol/kg/day, or any range between any of these values. In some embodiments, the therapeutically effective amount is dosed multiple times per day, such as two, three, four, or more times per day.

In certain embodiments, the therapeutically effective amount for treating, ameliorating, or preventing any physiological condition that can be treated, ameliorated, or prevented by oxytocin using the compounds of the disclosure is from 0.001 to 1000 nmol/kg/day. In certain embodiments, the therapeutically effective amount treating, ameliorating, or preventing any physiological condition that can be treated, ameliorated, or prevented by oxytocin using the compounds of the disclosure is about 0.001, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1000 nmol/kg/day, or any range between any of these values. In some embodiments, the therapeutically effective amount is dosed multiple times per day, such as two, three, four, or more times per day.

In certain embodiments, the therapeutically effective amount of a compound of the disclosure that is used to treat, ameliorate, or prevent any physiological condition that can be treated, ameliorated, or prevented by oxytocin is 2, 3, 4, 5, 10, 15, 20, 25, 30, 35, 40, 45, 50, 55, 60, 65, 70, 75, 80, 90, 100, 200, 300, 400, 500, 600, 700, 800, 900, 1000, 1100, 1200, 1300, 1400, 1500, 2000, 3000, 4000, 5000, 6000, 7000, 8000, 9000, 10000, 10⁵, or 10⁶-fold lower than the comparable therapeutic amount of oxytocin that would be used.

Dosing information derived from rat or mouse models can be readily converted into doses suitable for therapeutic use in humans. For example, and without wishing to be bound by the methodology described therein, doses obtained from or using mouse models can be converted to doses suitable for humans as described in Zhang H., et al. (2013) Treatment of Obesity and Diabetes Using Oxytocin or Analogs in Patients and Mouse Models. PLoS ONE 8(5): e61477. doi:10.1371/joumal.pone.0061477.

In certain embodiments, the I.U. of oxytocin corresponds to 1.67 μg oxytocin. In certain embodiments, the I.U. of oxytocin acetate corresponds to about 1.8 μg of oxytocin acetate. In certain embodiments, the molecular weight of oxytocin is 1,007.19 g/mole. In certain embodiments, the molecular weight of oxytocin acetate is 1,067.2 g/mole.

### Combination Therapies

The compounds useful within the methods described herein can be used in combination with one or more additional therapeutic agents useful for: treating, preventing, and/or ameliorating obesity; decreasing appetite or food intake; or modulating, treating, ameliorating, and/or preventing any other disease, disorder, and/or condition recited herein. These additional therapeutic agents may comprise compounds that are commercially available or synthetically accessible to those skilled in the art. These additional therapeutic agents are known to treat or reduce the symptoms of obesity, decrease appetite or food intake, or modulate, treat, ameliorate, and/or prevent any other disease, disorder, and/or condition recited herein.

In certain embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat obesity or decrease appetite or food intake in the patient. An effective amount of the therapeutic compound necessary to achieve a therapeutic effect may vary according to factors such as the state of the disease or disorder in the patient; the age, sex, and weight of the patient; and the ability of the therapeutic compound to treat obesity or decreasing appetite or food intake in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of obesity in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Certain embodiments of the present application can be better understood by reference to the following Examples which are offered by way of illustration. The scope of the present application is not limited to the Examples given herein.

### Materials and Methods

For studies utilizing a DIO rat model, male Sprague Dawley rats (CD-IGS rats, strain 001; approximately 4 weeks of age; 51-75 g) purchased from Charles River Laboratories (Wilmington, MA, USA). These rats were fed a diet of 60% calories from fat (Formula: D12492; Research Diets, Inc., New Brunswick, NJ, USA; 5.21 kcal/g) for 4.5 to 6 months prior to the start of the study to induce obesity. For studies utilizing lean rats, male Sprague Dawley rats (CD-IGS rats, strain 001; 226-250 g) purchased from Charles River Laboratories (Wilmington, MA, USA). All rats had ad libitum access to food and water and were kept on a 12 h light/12 h dark cycle. Animals were individually housed in a temperature (22±1° C.) and humidity (57±4%) controlled room. All body weight measurements were taken just prior to the start of the dark cycle.

### Design of ASK2131

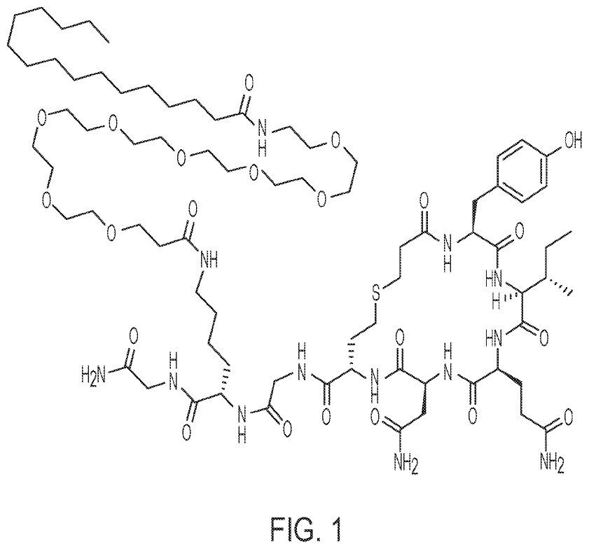

To improve OXTR agonist activity for the targeted molecule, enhance vasopressin receptor (V1aR) selectivity, and to increase its in vivo stability, two key modifications were introduced into ASK2131 (FIG. 1). First, the disulfide bridge —S—S— of ASK1476 was replaced with the respective thioether isostere and the Cys amino group was eliminated. Secondly, ⁷Pro was replaced with ⁷Gly to improve V1aR selectivity and ⁸Leu was changed to ⁸Lys followed by modification of terminal NH₂ with polyethylene glycol spacer and a lipid palmitoyl group.

### Chemical Synthesis of Test Articles

Oxytocin (OXT) was purchased from Sigma-Aldrich for use in in vitro functional studies (St. Louis, MO). Compound ASK2131 was synthesized by a hybrid Boc/Fmoc strategy on MBHA resin using approach described in. The following derivatives were employed: Boc-Gly-OH, FmocHcy((CH₂)₂COOtBu)-OH, Fmoc-Asn(Trt)-OH, Fmoc-Gln(Trt)-OH, Fmoc-Ile-OH and Boc-Tyr(tBu)-OH or Boc-Phe-OH. The Lys(PEG-palmitoyl) group was incorporated into the targeted molecule via Fmoc-Lys(Dde)-OH addition into the peptide followed by deprotection with hydrazine. Fmoc-NH-PEGs-COOH was added via solid-phase peptide synthesis and deprotected followed by addition of palmitic acid, C₁₅—COOH. The couplings were mediated by DIC/HOBt with a 3-fold excess of reagents.

The resin bound peptides were treated with the TFA/TIS/H2O 95/2.5/2.5 (v/v/v) mixture to remove the acid sensitive protecting groups and were subsequently cyclized with BOP/DIPEA in DMF. The crude cyclic peptide was cleaved from the resin with HF/anisole 10/1 (v/v) and purified by semi-preparative HPLC (Kinetex 5 mM, 100 A pore size, Biphenyl/TMS, 10×250 mm column). Mobile phases were as follows A: 0.05% aq. formic acid, B: 0.05% formic acid in MeCN; 1-10 min/1%-95% B gradient; retention time 5.94 min. The purity of the targeted molecule ASK2131 used for the subsequent in vitro and in vivo assays was 98.5%. HRMS (ESI) calculated for C₇₆H₁₃₁N₁₃O₂₂S: 1609.9252 (M+), found: 1609.9213. The molecular weight was 1611.

### In Vitro Functional Assay Measuring OXTR

Functional activity of ASK2131 at the OXT receptor was assessed using a Ca²⁺ flux fluorometric imaging plate reader assay. In a typical experimental protocol, stably transfected Chinese hamster ovary (CHO) hOTR NFAT bLac cells were plated at a density of 10,000 cells/well in a 384-well plate in growth medium (Dulbecco's modified Eagle's medium+Glutamax (Thermo Fisher Scientific, Waltham, MA), 10% dialyzed fetal bovine serum). The next day calcium-sensitive, fluo-4 dye (Invitrogen, Carlsbad, CA) and probenecid (Invitrogen; 2.5 mM) was dissolved in 20 ml warm dye loading buffer (Hanks' balanced salt solution with calcium and magnesium, 20 mM Hepes, pH 7.4) and added to each well. Plates were incubated for 60-120 minutes in a 37° C. cell incubator to allow cells to uptake the dye. The control peptide (OXT) and ASK2131 were diluted in Hanks' balanced salt solution with calcium and magnesium, 20 mM HEPES, and 0.1% bovine serum albumin. After dye-loading, the cell plates were placed on the fluorometric imaging plate reader. A total of 15 ml of test compound was added to the 30 ml of dye on the cells, and intracellular Ca²⁺ was monitored for 90 seconds. Intracellular Ca²⁺ concentrations were calculated as the maximum increase over basal levels. Responses were normalized to control responses. Data were analyzed by curve fitting.

### In Vitro Functional Assay Measuring V₁ₐR

Evaluation of the agonist activity of OXT and ASK2131 at the human V1a receptor was conducted using stably transfected CHO cells by measuring the test article effect on cytosolic Ca²⁺ ion mobilization via a fluorimetric detection method. The cells were suspended in DMEM buffer (Invitrogen, Carlsbad, CA) complemented with 0.1% FCSd, then distributed in microplates at a density of 4.5×10⁴ cells/well. The fluorescent probe (Fluo4 Direct, Invitrogen, Carlsbad, CA) mixed with probenicid in HBSS buffer (Invitrogen, Carlsbad, CA) complemented with 20 mM HEPES (Invitrogen, Carlsbad, CA) (pH 7.4) was then added into each well and equilibrated with the cells for 60 min at 37° C. then 15 min at 22° C. The assay plates were positioned in a microplate reader (CellLux, PerkinElmer) followed by the addition of the test compound ASK2131, reference agonist or HBSS buffer (basal control), and the measurements of changes in fluorescence intensity which varies proportionally to the free cytosolic Ca² ion concentration. For stimulated control measurements, AVP at 1 μM was added in separate assay wells. The results were expressed as a percent of the control response to 1 μM AVP. The standard reference agonist was AVP, which was tested in each experiment at several concentrations to generate a concentration-response curve from which its EC₅₀ value was calculated.

In Vitro Functional Assay measuring V₂R Evaluation of the agonist activity of ASK2131 at the human V₂ receptor expressed in stably transfected CHO cells was conducted by measuring its effects on cAMP production using the HTRF detection method. The cells were suspended in HBSS buffer (Invitrogen, Carsbad, CA) complemented with 20 mM HEPES (pH 7.4), 0.010% BSA and 500 μM IBMX, then distributed in microplates at a density of 3.104 cells/well and incubated for 30 min at room temperature in the absence (control) or presence of the test compound or the reference agonist.

For stimulated control measurement, separate assay wells contained 1 nM AVP. Following incubation, the cells were lysed, and the fluorescence acceptor (D2-labeled cAMP) and fluorescence donor (anti-cAMP antibody labeled with europium cryptate) were added. After 60 min at room temperature, the fluorescence transfer was measured at γ ex=337 nm and γ em=620 and 665 nm using a microplate reader (Envision, Perkin Elmer). The cAMP concentration was determined by dividing the signal measured at 665 nm by that measured at 620 nm (ratio).

The results were expressed as a percent of the control response to 1 nM AVP. The standard reference agonist was AVP, which was tested in each experiment at several concentrations to generate a concentration-response curve from which its EC₅₀ value was calculated.

### Preparation and Administration of Drug

A variety of different protocols were tested for getting the lyophilized peptide products into solution for in vivo testing, including a 10% self-emulsifying drug delivery system (SEDDs): 90% 20 mM, pH 5, phosphate buffer (v/v) (SEDDs is 3:4:3 Miglyol 812: Cremophor RH40: Capmul).

Surprisingly and unexpectedly, it was discovered that, in some embodiments, cyclodextrins were particularly effective at achieving the best and most consistent weight reducing effects when combined with the compounds of formula (I). In certain embodiments, hydroxypropyl-β-cyclodextrin excipient (HP-CD) was used. ASK2131 stock solution was prepared using a 10:1 (wt:wt) HP-CD excipient [(HP-CD 1541 g/mol)/(ASK2131 1611 g/mol)] ratio in sterile ultra-pure H₂O and gently agitated at 4° C. for >24 h. This excipient has been tested previously to ensure that it does not illicit anorectic effects independently. The stock was aliquoted and stored at −20° C. Oxytocin (Syntocinon) was purchased from Selleck Chemical (cat #P1029; lyophilized) and dissolved in 1 mL if sterile ultra-pure H₂O. The stock was aliquoted and stored at −20° C. Stock solutions for both drugs were diluted to working solution in 0.9% normal saline solution and mixed gently prior to use. Normal saline solution was used as the vehicle control in all studies.

Vehicle, native OXT, and ASK2131 were injected once daily at the start of the dark cycle using subcutaneous injection with a 1 cc 29 G insulin syringe. The concentration of both OXT and ASK2131 solutions were adjusted so that dosing volume remained at 1 mL/kg.

### Pharmacokinetics Study

Male lean SpragueDawley rats (~300 g; n=5 per group) were administered 300 nmol/kg dose of either ASK2131 or OXT via sc injection to assess absorption, peak concentration, and clearance. Serial blood samples (200 μL per time point) were obtained via tail nick using K3EDTA microvettes (mfg. #20.1288.100; Sarstedt, Inc., Numbrecht, Germany) prior to and 1, 2, 4, 6, 12, and 24-h post-drug administration. Whole blood samples were immediately centrifuged at 4° C.; separated plasma (~100 μL) was pipetted into micro centrifuge tubes, flash frozen on dry ice, and stored at −80° C. Quantitative analysis plasma samples were performed by Alturas Analytics (Moscow, ID, USA) via peptide extraction from acidified plasma with acetonitrile followed by LC-MS/MS analysis.

### Long-Term Drug Intervention, Blood Parameters, and Gene Expression

Male Sprague Dawley rats were fed 60% HF diet (D12492; Research Diets, Inc; New Brunswick, NJ; 5.21 kcal/g) for 16 weeks prior to the start of the study. Rats were then singly house in BioDAQ cages (Research Diets, Inc.; New Brunswick, NJ) and allowed to acclimate to their new environment for 10 days. Baseline measures of body weight, food intake and water intake were taken for one week to balance the groups and create feeding pairs. Each group (n=6 animals/group) averaged 714.47±81.6 g at baseline (Table 3).

Effects of ASK2131 were compared to a vehicle-treated group, OXT-treated group, as well as a pair-fed control group. Three days of baseline measures were taken before subcutaneous treatment administration began on day 4 of the study. Assessments were made to body weight, food intake and water intake. ASK2131 was administered at 50 nmol/kg throughout the entire study period. OXT doses were titrated during the first 6 days of the study. OXT-treated animals were started at 50 nmol/kg dose for two days, followed by 150 nmol/kg, then 300 nmol/kg, until ending at 600 nmol/kg for the remainder of the study.

Cardiac puncture was used to collect blood for analysis 2 h post final injection. Commercially available enzyme-linked immunosorbent assays (ELISA) were used for quantitative assessment of leptin (cat. #EZRL-83K; MilliporeSigma, Burlington, MA, USA), insulin (cat. #EZRMI-13K; MilliporeSigma, Burlington, MA, USA), and adiponectin (cat. #EZRAPD-62K MilliporeSigma, Burlington, MA, USA). Serum samples were diluted 1:500 for the adiponectin ELISA using the sample diluent provided with the kit. All ELISAs were completed following the manufacturers recommendations. Levels of glucose, cholesterol (total, HDL, and calculated LDL), triglycerides, ALT, and AST were determined in the serum on a Modular P chemistry analyzer (Roche Diagnostics, Risch-Rotkreuz, Switzerland) by the University of Washington Nutrition and Obesity Research Center's Analytic Core (Seattle, WA, USA).

IWAT and IBAT were collected from the DIO rats treated with vehicle, oxytocin, ASK2131, and the pair-fed to the ASK2131. IWAT and IBAT were homogenized using a TissueLyser LT (Qiagen, Hilden, Germany). RNA was extracted from the tissue lysates using the RNeasy Lipid Mini Kit (mfg. #74804; Qiagen, Hilden, Germany) and reverse transcribed using the high-capacity cDNA kit (mfg. #4368814; Applied Biosystems, Waltham, MA, USA). qRT-PCR was completed on the mRNA samples using the Applied Biosystems StepOnePlus System (Applied Biosystems, Waltham, MA, USA). All samples were normalized to the cycle threshold value of 18S mRNA. The following TaqMan® probes (Applied Biosystems, Waltham, MA, USA) were used in this study: 18S (cat. #Hs99999901_s1), UCP1 (cat. #Rn00562126_m1), and Cox8b (cat. #Rn00562884_m1). Analysis was completed using the delta CT method.

A secondary analysis was performed comparing the first 22 days of ASK1476 and OXT treatment from our reported experiment with ASK2131 and OXT in the current 22-day experiment. Analyses were limited to body weight change and differences in cumulative food intake.

### Dose Escalation Study

Male DIO Sprague Dawley rats (n=4) were fed 60% HF diet (formula: D12492; Research Diets, Inc., New Brunswick, NJ, USA; 5.21 kcal/g) for 16 weeks prior to the start of the study. Rats were changed to being singly housed and allowed to acclimate for at least one week prior to the start of this study. Animals averaged 844.15 Z 99.95 g at baseline. Baseline measures of body weight, food intake, and water intake were taken for 6 days before drug administration began. Doses for the dose escalation study included 1, 2, 5, 10, 20, 50, and 100 nmol/kg. Body weight, food and water intake were assessed just prior to the start of the dark cycle. Surprisingly and unexpectedly, the dose escalation studies presented here show the remarkable differences between ASK2131 and ASK1476. In certain embodiments, the greatest anorectic effect achieved with ASK1476 occurred at the 300 nmol/kg/d dose yielding a ~34% reduction in caloric intake while ASK2131 dosed at 2 nmol/kg/d yielded a ~55% reduction in caloric intake. An even more stark contrast can be seen considering that OXT, which reached a maximum effect at 1200 nmol/kg/d, yielded a ~36% reduction in caloric intake.

### Statistical Analyses

Statistical analyses were performed using GraphPad Prism 9 Software (La Jolla, CA, USA. Two-way ANOVAs were used for comparison of repeated measures with Šidák's multiple comparison test for post hoc analyses. For cross-sectional comparisons of normally distributed measures taken at only one time point, an ordinary one-way ANOVA was used when group variances are assumed equal, and Welch's ANOVA was used for unequal variances. Post hoc comparisons between groups were made using Šidák's multiple comparison test. Skewed data were analyzed using a nonparametric test, i.e., Kruskal-Wallis test assuming equal variances or Brown-Forsythe ANOVA assuming unequal variances; an F-test was used to compare variances between groups and post hoc comparisons were made using Dunn's or Dunnett's T3 multiple comparison tests as appropriate. In all instances, a p<0.05 was considered significant. All values are represented as mean (SEM).

### Results

### In Vitro Functional Assay

The design of ASK2131 was based on recent reports describing OXT receptor agonists with extended in vivo stability. Specifically, carba hexapeptide core was prepared by replacing one S atom of the disulfide bridge with respective CH₂ group. Pro⁷ was changed to Gly⁷ to amend functional activity of the resulting molecule at the vasopressin Via receptor (V1aR) while maintaining its OXTR agonistic potency. Leu⁸ was replaced with Lys⁸ and the terminal NH group of the Lys⁸ was lipidated to enhance binding of the molecule to plasma proteins in vivo. This was expected to reduce the free fraction of ASK2131 available for enzymatic degradation. Receptor pharmacology profile of both OXT and ASK2131 against OXTR, V1aR and V₂R are summarized in Table 1.

TABLE 1
Receptor pharmacology profile of both OXT
and ASK2131 against OXTR, V₁ₐR and V₂R.

| | hOXTR, | hV₁ₐR, | hV₂R, | Ratio OXTR/ |
|---|---|---|---|---|
| Compound | EC₅₀ (nM) | EC₅₀ (nM) | EC₅₀ (nM) | V₂R/ |
| OXT [1] | 2.3 | 10 | 7.3 | 0.32 |
| ASK2131 | 1.1 | NA (>1,000) | 0.36 | 3.06 |

Notably, ASK2131 did not display V1aR activation whereas its agonist activity for OXTR was 1.1 nM. Furthermore, the molecule was potent activator of V₂R to show EC₅₀ of 0.36 nM in the cAMP assay.

### Pharmacokinetics

Pharmacokinetics data for OXT and ASK2131 following intravenous (iv) and subcutaneous (sc) administration to rats are summarized in Table 2. Following iv administration in rats, the lipidated analogue ASK2131 exhibited considerably reduced plasma clearance (CLₚₗₐₛₘₐ=1.2 mL/mg/kg) and an increased steady-state volume of distribution (V_{d}=0.80 L/kg) compared to OXT, resulting in a half-life (T/2) of 2.3 hours and Tₘₐₓ of 4 hours. The lipidated analog ASK2131 also displayed a total maximal plasma concentration (Cₘₐₓ) of 276 nM and free Cₘₐₓ of 0.6 nM at 4 hours post-dose further suggesting increased plasma binding profile of the molecule. The area under the concentration-time curve from dosing (time 0) to the time of the last measured concentration (AUClast) was 1.95 hr*mg/mL suggesting good plasma stability and relevant exposure for subsequent efficacy studies.

TABLE 2
Pharmacokinetic parameters for OXT [2] and
ASK2131 following intravenous (iv) and subcutaneous
(sc) administration to rats and mice.

| Compound, | CLₚₗₐₛₘₐ, | V_{d}, | Cₘₐₓ/free | Tₘₐₓ, | T_{1/2}, |
|---|---|---|---|---|---|
| route | mL/min/kg | L/kg | Cₘₐₓ, nM | hrs | hrs |
| OXT, iv | 17.1 | 0.085 | | | 0.12 |
| (rat) [2] | | | | | |
| OXT, sc | | | 216/191 | 0.25 | 0.5 |
| (mouse) [2] | | | | | |
| ASK2131, | 1.20 | 0.80 | 276/0.5 | 4.00 | 2.30 |
| iv (rat) | | | | | |

### Body Weight, Food Intake, and Metabolic Outcomes in Long-Term Study

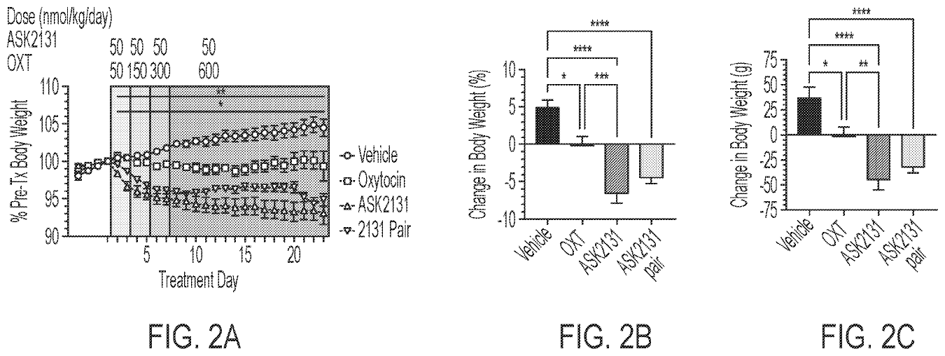

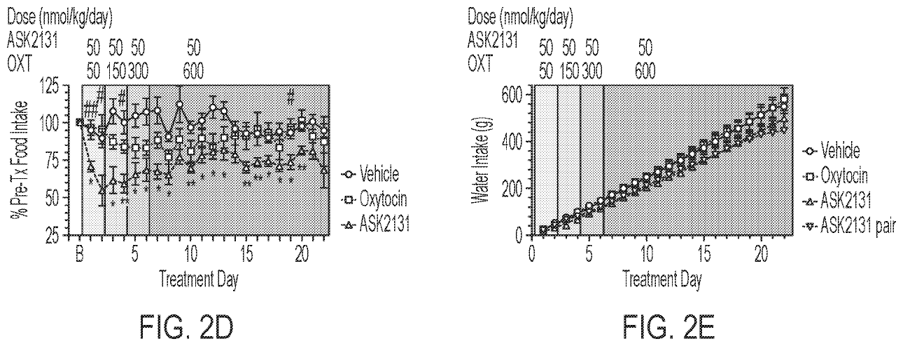

After 2 days of drug administration, ASK2131-treated animals showed a significant decrease in body weight compared to the vehicle-treated group (predicted mean difference (PMD)−3.83%; 95% CI−6.52 to −1.15%; p=0.0011) and the OXT-treated group (PMD−3.77%; 95% CI−6.45 to −1.08%; p=0.0014). This was maintained until study completion leading to a body weight reduction of 11.5% over vehicle treatment (FIGS. 2A-C). On treatment day 9 of the study, after 8 days of drug administration, OXT-treated animals showed a significant difference in body weight compared to the vehicle-treated group (PMD −3.12%; 95% CI−5.80 to −0.43%; p=0.014) leading to a difference in weight change of 5% weight loss vs. vehicle at the end of treatment. Pair-fed ASK2131 treated animals were given the same amount of food as their paired ASK2131 treated animal.

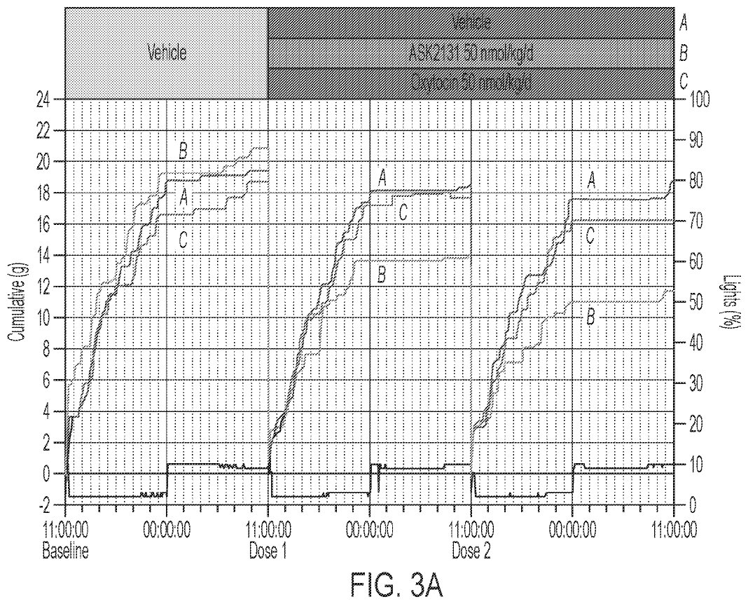

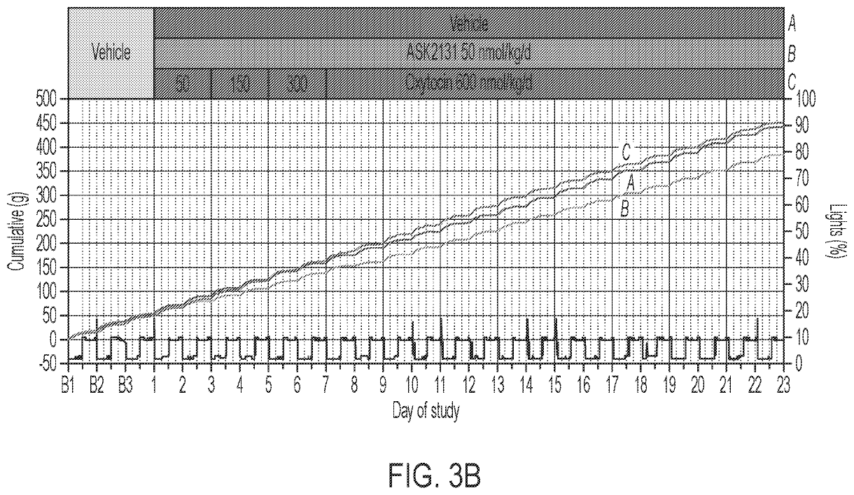

At treatment day 20 of the study, after 19 days of pair feeding, the ASK2131 pair-fed group showed significant body weight reduction compared to the ASK2131-treated group (PMD−2.90%; 95% CI−5.58 to −0.21%; p=0.027) (FIG. 2A). Analysis of food intake pattern showed a consistent and stronger reduction of food intake starting at day 1 of treatment compared to OXT with comparable doses (50 nmol/kg/d) (FIGS. 2D, 3A) with a 39±9% reduction of food intake at day 3 compared to baseline and pronounced reduction of food intake compared vehicle-treated group (at day 3: PMD −45.99%; 95% CI−80.47 to −11.51%; p=0.0102). Even when doses were increased for OXT every 2 days up to 600 nmol/kg while maintaining the dose for ASK2131 at 50 nmol/kg/d, food intake reduction was more pronounced in response to ASK2131 vs. OXT as demonstrated by daily food intake (FIG. 2D) and cumulative food intake (FIG. 31B). OXT-animals did not exhibit a significant reduction in food intake compared to the vehicle-treated group (95% CI 0.2550 to 40.59; p=0.0464).

Water intake was significantly reduced compared to vehicle only on the second day of treatment with ASK2131 at a time when the strongest effect of food intake reduction was noted (95% CI 0.8276 to 67.46; p=0.0422). All other groups exhibited no significant differences in water intake both compared to baseline or other treatment groups (FIG. 2E).

DIO Sprague-Dawley rats were administered treatment for 22 days. At the time of euthanasia, blood samples were collected through cardiac puncture. Compared to vehicle, ASK2131-treated rats showed significant decreases in cholesterol (95% CI 0.075 to 51.92; p=0.0493), HDL (95% CI 0.7079 to 16.89; p=0.0318), and LDL (95 CI 0.6800 to 37.92; p=0.0415) (Table 3). No significant differences were observed in terms of glucose, triglyceride, ALT, and AST levels. Leptin, adiponectin, and insulin were measured in these animals. No significant difference was observed between the groups (Table 3).

TABLE 3

| | | | | ASK2131 |
|---|---|---|---|---|
| | Vehicle | Oxytocin | ASK2131 | Pair |
| N= | 6 | 6 | 6 | 6 |
| Pre- | 762 ± 64 | 719 ± 18 | 723 ± 38 | 724 ± 43 |
| treatment | | | | |
| body | | | | |
| weight (g) | | | | |
| Post- | 799 ± 69 | 719 ± 20 | 676 ± 42 | 691 ± 41 |
| treatment | | | | |
| body | | | | |
| weight (g) | | | | |
| % change in | 4.9 ± 1.1 | 0.1 ± 1 ^{b} | −6.6 ± 1.3 ^{c, d} | −4.6 ± 0.6 |
| body weight | | | | |
| Glucose | 232 ± 8 | 203 ± 13 | 237 ± 12 | 256. ± 13 |
| (mg/dL) | | | | |
| Triglycerides | 190 ± 10 | 145 ± 7 | 182 ± 30 | 128 ± 7 |
| (mg/dL) | | | | |
| Cholesterol | 102 ± 8 | 76 ± 3 | 67 ± 6 ^{b} | 73 ± 6 ^{a} |
| (mg/dL) | | | | |
| HDL | 28 ± 3 | 25 ± 2 | 21 ± 2 | 23 ± 2 |
| (mg/dL) | | | | |
| LDL | 31 ± 5 | 22 ± 2 | 15 ± 5 | 24 ± 5 |
| (mg/dL) | | | | |
| * calculated | | | | |
| ALT (U/L) | 44 ± 13 | 40 ± 5 | 26 ± 2 | 28 ± 3 |
| AST (U/L) | 117 ± 30 | 128 ± 22 | 73 ± 8 | 100 ± 8 |
| Leptin | 55.5 ± 11.1 | 54.9 ± 7.9 | 39.0 ± 9.1 | 37.8 ± 8.0 |
| (mg/dL) | | | | |
| Insulin | 5.6 ± 1.5 | 7.2 ± 2.1 | 6.7 ± 1.0 | 10.2 ± 2.6 |
| (ng/mL) | | | | |
| Adiponectin | 76.2 ± 2.7 | 60.8 ± 3.9 | 62.4 ± 7.3 | 48.2 ± 6.4 |
| (ng/mL) | | | | |

^{a} p < 0.05 vs. vehicle
^{b} p < 0.01 vs. vehicle
^{c} p < 0.001 vs. vehicle
^{d} p < 0.001 vs. OXT.
Data expressed as mean ± SEM. Primary Analysis for normal distribution: One-Way ANOVA test assuming equal variances or Welch's ANOVA for unequal variances; Šídák's multiple comparisons test was used for post hoc analyses in both instances. For non-normally distributed data: Kruskal-Wallis test with Dunn's multiple comparison test assuming equal variances or Brown-Forsythe ANOVA test with Dunnett's T3 multiple comparison test for unequal variances. Serum measures were only available for n = 4 of the Oxytocin treated animals.
* Calculated LDL = Cholesterol − (HDL + Triglycerides/5).

### Body Weight and Food Intake in Dose Escalation Study

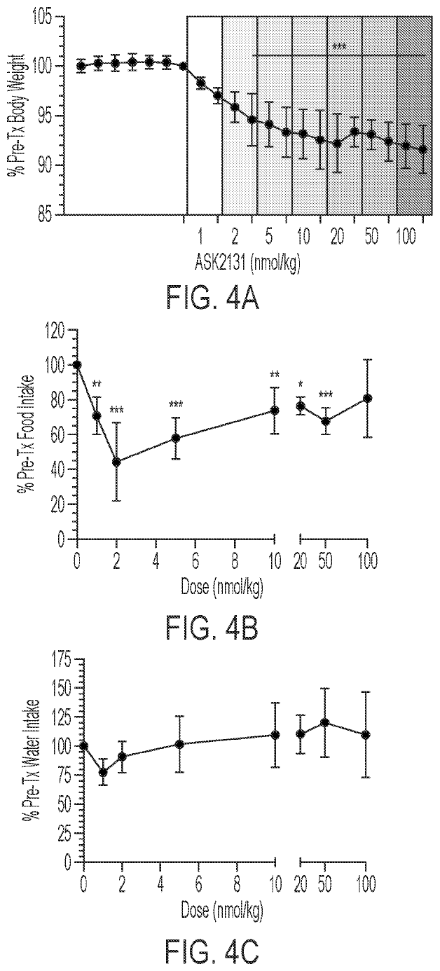

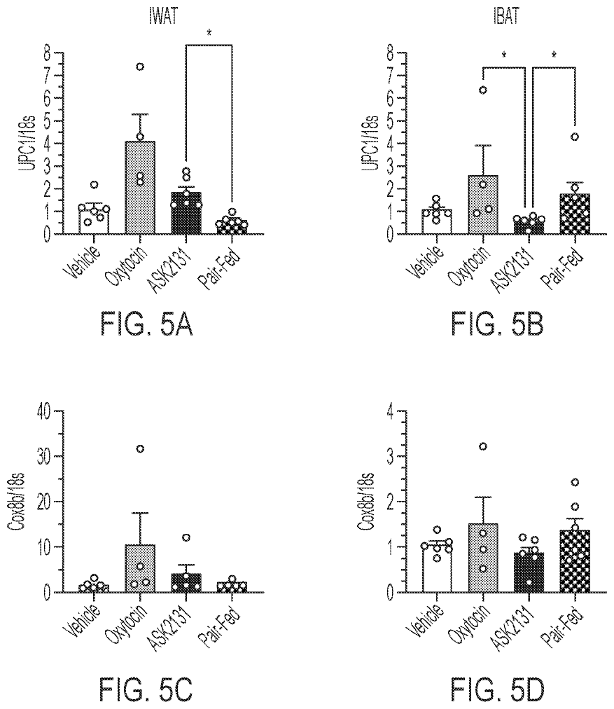

To test the range of responses to different drug doses, DIO Sprague-Dawley rats were administered increasing doses of ASK2131 every two days start at 1 nmol/kg and ending at 100 nmol/kg. Body weight showed continuous decline over the course of the treatment period and on average each rat showed an 8.4% loss in body weight compared to baseline (FIG. 4A). Food intake reduction showed a U-shaped response. The highest level of reduction (~47%) was observed at 2 nmol/kg. At doses <2 nmol/kg and >5 nmol/kg, food intake was reduced approximately 20-25% (FIG. 4B). Water intake showed a modest decline with treatment onset but was not significantly different from baseline with increased dosing of ASK2131 (FIG. 4C).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### ENUMERATED EMBODIMENTS

The following enumerated embodiments are provided, the numbering of which is not to be construed as designating levels of importance:

Embodiment 1 provides a compound of formula (I), or a pharmaceutically acceptable salt, enantiomer, diastereomer, or solvate thereof:

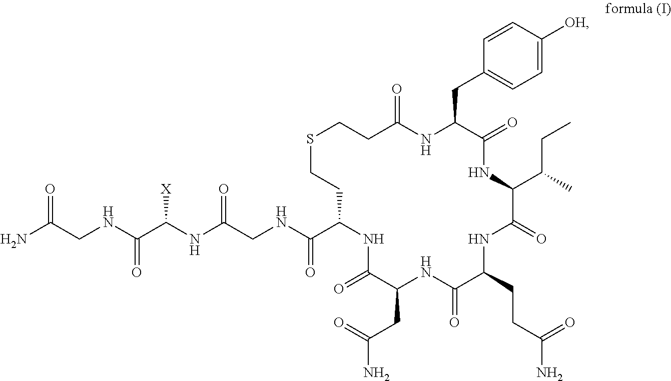

wherein:
X has the structure

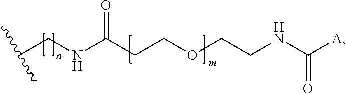

A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

Embodiment 2 provides the compound of embodiment 1, wherein n is 2, 3, or 4.

Embodiment 3 provides the compound of any one of embodiments 1-2, wherein n is 4.

Embodiment 4 provides the compound of any one of embodiments 1-3, wherein m is 4, 5, 6, 7, 8, 9, or 10.

Embodiment 5 provides the compound of any one of embodiments 1-4, wherein m is 8.

Embodiment 6 provides the compound of any one of embodiments 1-5, wherein A is C₁₀₋₂₀ alkyl.

Embodiment 7 provides the compound of any one of embodiments 1-6, wherein A is C₁₅ alkyl.

Embodiment 8 provides the compound of any one of embodiments 1-7, wherein the compound is

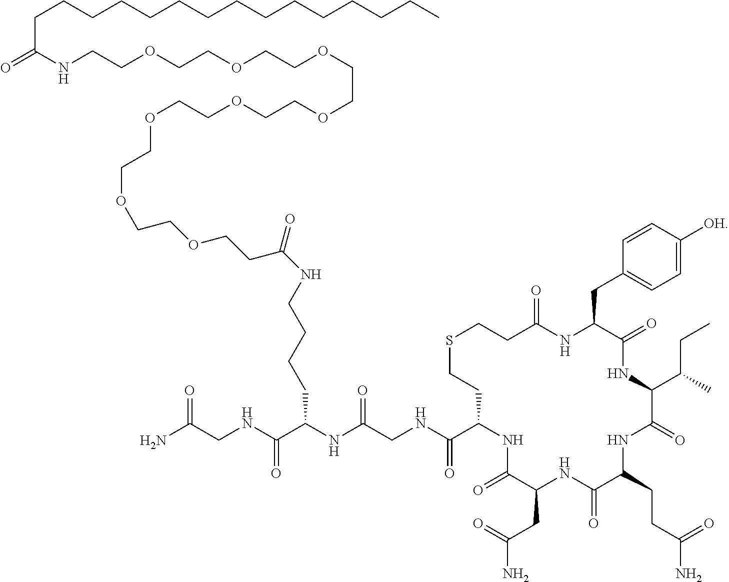

Embodiment 9 provides a composition comprising:
at least one cyclodextrin, and
a compound of formula (I), or a pharmaceutically acceptable salt, enantiomer, diastereomer, or solvate thereof:

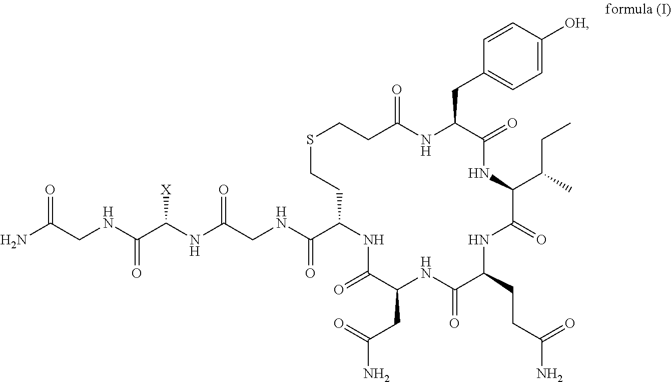

wherein:
X has the structure

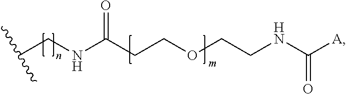

A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

Embodiment 10 provides the composition of embodiment 9, wherein the at least one cyclodextrin and the compound are in a ratio of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or about 20:1 (wt/wt).

Embodiment 11 provides the composition of any one of embodiments 9-10, wherein the at least one cyclodextrin comprises hydroxypropyl-β-cyclodextrin.

Embodiment 12 provides the composition of any one of embodiments 9-11, further comprising at least one additional pharmaceutically acceptable excipient or carrier.

Embodiment 13 provides composition of any one of embodiments 9-12, which is a solution.

Embodiment 14 provides a method of treating, preventing, and/or ameliorating in a subject, the method comprising administering to the subject a therapeutically effective amount of a compound of formula (I), or a pharmaceutically acceptable salt, enantiomer, diastereomer, or solvate thereof:

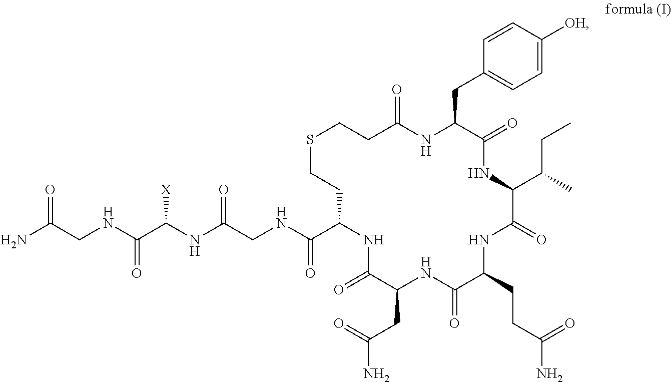

wherein:
X has the structure

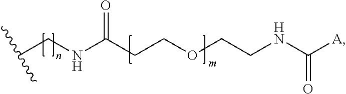

A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

Embodiment 15 provides the method of embodiment 14, wherein administering is by a route of administration selected from the group consisting of oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 16 provides the method of any one of embodiments 14-15, wherein the subject is human.

Embodiment 17 provides the method of any one of embodiments 14-15, wherein the compound is formulated as a pharmaceutical composition.

Embodiment 18 provides the method of any one of embodiments 14-17, wherein the composition is an aqueous solution.

Embodiment 19 provides the method of any one of embodiments 14-18, further comprising administering at least one additional agent to the subject.

Embodiment 20 provides the method of any one of embodiments 14-19, wherein the composition comprises at least one cyclodextrin.

Embodiment 21 provides the method of any one of embodiments 14-20, wherein the compound is

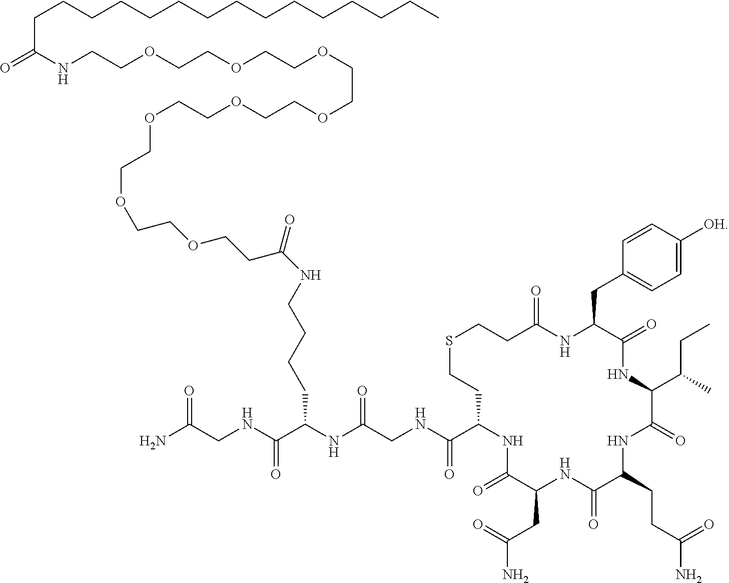

Embodiment 22 provides a method of reducing appetite or food intake in a subject, the method comprising administering to the subject a therapeutically effective amount of a compound of formula (I), or a pharmaceutically acceptable salt, enantiomer, diastereomer, or solvate thereof:

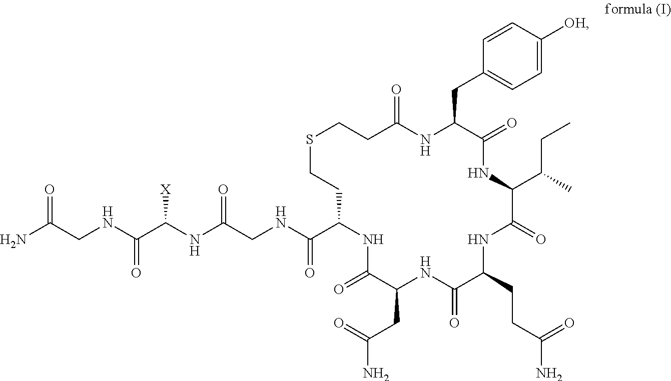

wherein:
X has the structure

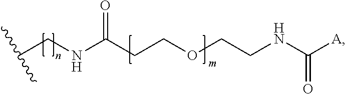

A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

Embodiment 23 provides the method of embodiment 22, wherein administering is by a route of administration selected from the group consisting of oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 24 provides the method of any one of embodiments 22-23, wherein the subject is human.

Embodiment 25 provides the method of any one of embodiments 22-24, wherein the compound is formulated as a pharmaceutical composition.

Embodiment 26 provides the method of any one of embodiments 22-25, wherein the composition is an aqueous solution.

Embodiment 27 provides the method of any one of embodiments 22-26, further comprising administering at least one additional agent.

Embodiment 28 provides the method of any one of embodiments 22-27, wherein the composition comprises at least one cyclodextrin.

Embodiment 29 provides the method of any one of embodiments 22-28, wherein the compound is

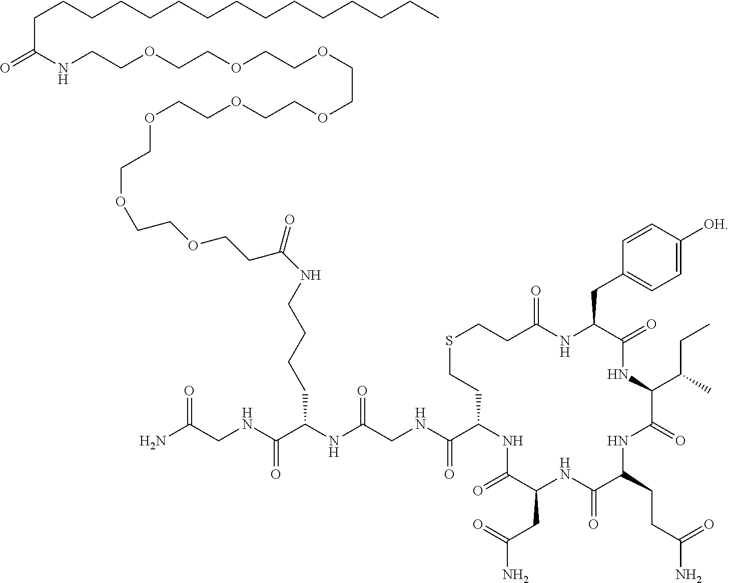

1. A compound of formula (I), or a pharmaceutically acceptable salt, enantiomer, diastereomer, or solvate thereof: 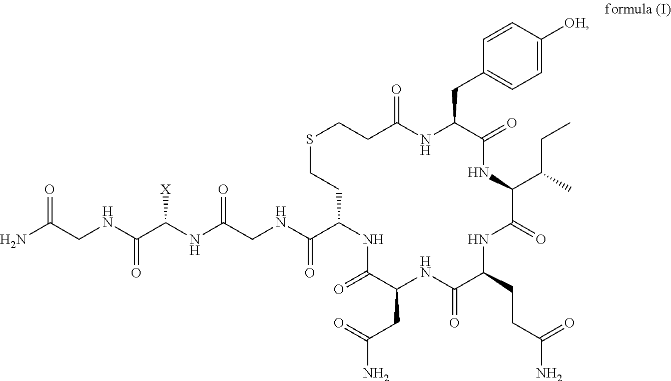
wherein:
X has the structure 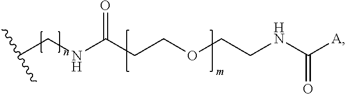
A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

2. The compound of claim 1, wherein n is 2, 3, or 4.

3. (canceled)

4. The compound of claim 1, wherein m is 4, 5, 6, 7, 8, 9, or 10.

5. (canceled)

6. The compound of claim 1, wherein A is C₁₀₋₂₀ alkyl.

7. The compound of claim 6, wherein A is C₁₅ alkyl.

8. The compound of claim 1, wherein the compound is

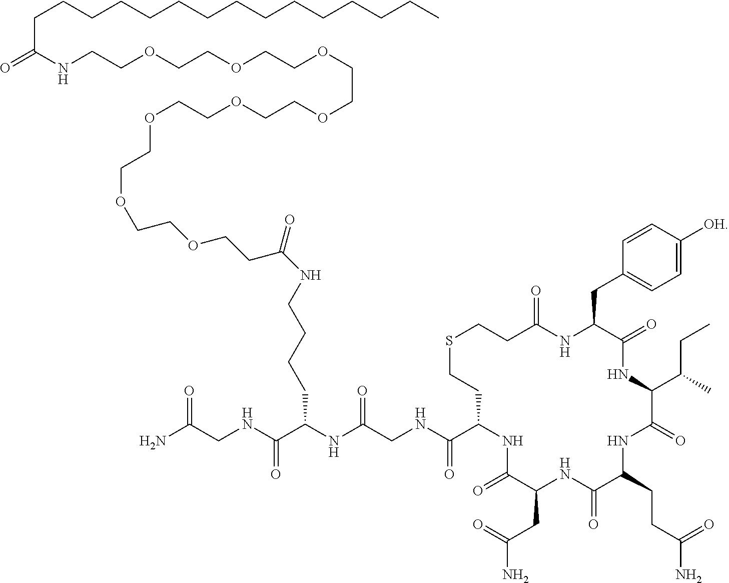

9. A composition comprising:
at least one cyclodextrin, and
a compound of formula (I), or a pharmaceutically acceptable salt, enantiomer, diastereomer, or solvate thereof: 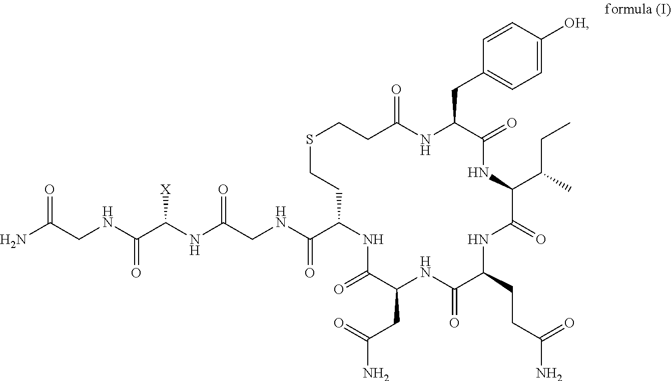
wherein:
X has the structure 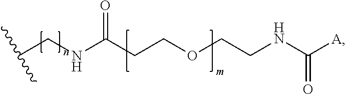
A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20.

10. The composition of claim 9, wherein at least one of the following applies:
i) the at least one cyclodextrin and the compound are in a ratio of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or about 20:1 (wt/wt);
ii) the at least one cyclodextrin comprises hydroxypropyl-β-cyclodextrin;
iii) the composition further comprises at least one additional pharmaceutically acceptable excipient or carrier; and
iv) the composition is a solution.

11-13. (canceled)

14. A method of treating, preventing, or ameliorating obesity in a subject, the method comprising administering to the subject a therapeutically effective amount of a compound of formula (I), or a pharmaceutically acceptable salt, enantiomer, diastereomer, or solvate thereof: 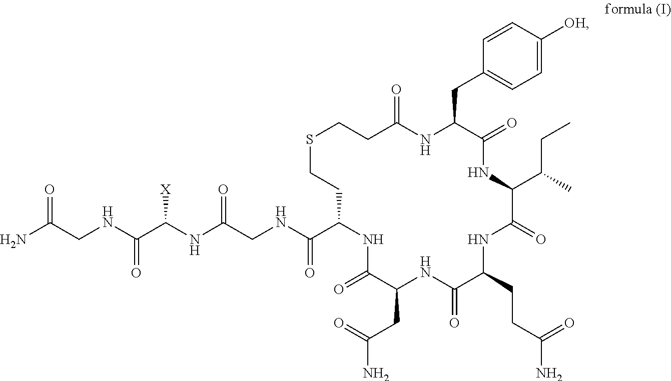
wherein:
X has the structure 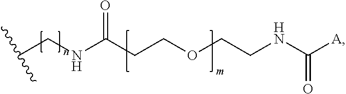
A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20;
wherein the compound is optionally formulated as a pharmaceutical composition comprising at least one pharmaceutically acceptable carrier or excipient.

15. The method of claim 14, wherein administering is by a route of administration selected from the group consisting of oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

16-17. (canceled)

18. The method of claim 14, wherein the composition is an aqueous solution.

19. The method of claim 14, further comprising administering at least one additional agent to the subject.

20. The method of claim 14, wherein the composition comprises at least one cyclodextrin.

21. The method of claim 14, wherein the compound is

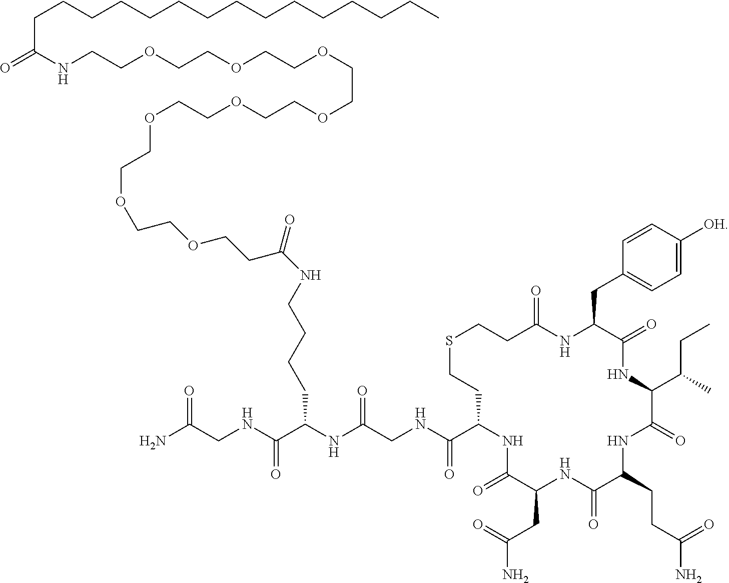

22. A method of reducing appetite or food intake in a subject, the method comprising administering to the subject a therapeutically effective amount of a compound of formula (I), or a pharmaceutically acceptable salt, enantiomer, diastereomer, or solvate thereof: 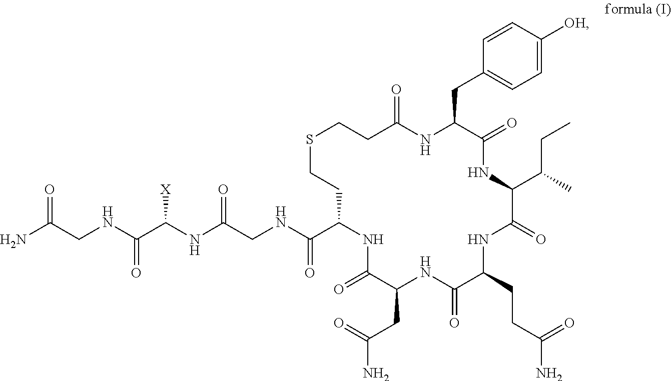
wherein:
X has the structure 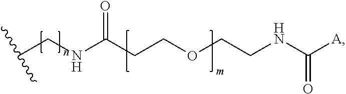
A is C₆₋₂₅ alkyl;
n is 1, 2, 3, 4, 5, or 6; and
m is 2, 3, 4, 5, 6, 7, 8, 9, 10, 11, 12, 13, 14, 15, 16, 17, 18, 19, or 20;
wherein the compound is optionally formulated as a pharmaceutical composition comprising at least one pharmaceutically acceptable carrier or excipient.

23. The method of claim 22, wherein administering is by a route of administration selected from the group consisting of oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

24-25. (canceled)

26. The method of claim 22, wherein the composition is an aqueous solution.

27. The method of claim 22, further comprising administering at least one additional agent.

28. The method of claim 22, wherein the composition comprises at least one cyclodextrin.

29. The method of claim 22, wherein the compound is [chemical structure]
